(19) United States
(12) Patent Application Publication
(10) Pub. No.: US 2001/0031262 A1
(43) Pub. Date: Oct. 18, 2001

(54) Controlled delivery of antigens

(76) Inventors: Michael Caplan, Woodbridge, CT (US); Gary A. Bannon, Little Rock, AR (US); A. Wesley Burks JR., Little Rock, AR (US); Hugh A. Sampson, Larchmont, NY (US)

Correspondence Address: Patrea L. Pabst, Arnall Golden & Gregory, LLP, 2800 One Atlantic Center, 1201 West Peachtree Street, Atlanta, GA, 30309-3450, US

(21) Appl. No.: 09/730,921

(22) Filed: Dec. 6, 2000

Related U.S. Application Data
(60) Provisional application No. 60/169,330, filed on Dec. 6, 1999.

(51) Int. Cl. A61K 39/395; A61K 9/127

(52) U.S. Cl. 424/178.1; 424/450

(57) ABSTRACT

Formulations and methods have been developed for delivering antigens to individuals in a manner that substantially reduces contact between the antigen and IgE receptors displayed on the surfaces of cells involved in mediating allergic responses, which target delivery of antigen to dendritic and other phagocytic APCs, and which have improved pharmacokinetics. By reducing direct and indirect association of antigens with antigen-specific IgE antibodies, the risk of an allergic reaction, possibly anaphylatic shock, is reduced or eliminated. Particularly preferred antigens are those that may elicit anaphylaxis in individuals, including food antigens, insect venom and rubber-related antigens. In the preferred embodiments, the compositions include one or more antigens in a delivery material such as a polymer, in the form of particles or a gel, or lipid vesicles or liposomes, any of which can be stabilized or targeted to enhance delivery. Preferably, the antigen is surrounded by the encapsulation material. Alternatively or additionally, the antigen is displayed on the surface of the encapsulation material. One result of encapsulating antigen is the reduction in association with antigen-specific IgE antibodies. In some embodiments, antigens are stabilized or protected from degradation until the antigen can be recognized and endocytized by APCs which are involved in elicting cellular and humoral immune responses. In a preferred embodiment, the formulation is designed to deliver antigens to individuals in a manner designed to promote a Th1-type mediated immune response and/or in a manner designed to suppress a Th2 response. In still another embodiment, the formulation effects preferential release of the antigen within APCs.

### BACKGROUND OF THE INVENTION

The present invention is generally in the area of controlled delivery of antigens for use in vaccination or to induce tolerance to allergens, and in particular relates to polymeric encapsulated formulation.

This application claims priority to U.S. Ser. No. 60/169,330 filed Dec. 6, 1999.

Allergies and Asthma

Allergic reactions pose serious public health problems worldwide. Pollen allergy alone (allergic rhinitis or hay fever) affects about 10-15% of the population, and generates huge economic costs. For example, reports estimate pollen allergy generated $1.8 billion of direct and indirect expenses in the United States in 1990 (Fact Sheet, National Institute of Allergy and Infectious Diseases; McMenamin, Annals of Allergy 73:35, 1994). Asthma, which can be triggered by exposure to antigens, is an even more serious disease, and can lead to death in extreme cases. Asthma currently accounts for millions of visits yearly to hospitals and is increasing in frequency. The only treatment currently available is for alleviation of symptoms, for example, to relieve constriction of airways.

More serious than the economic costs associated with pollen and other inhaled allergens (e.g., molds, dust mites, animal danders) is the risk of anaphylactic reaction observed with allergens such as food allergens, insect venoms, drugs, and latex.

Allergic reactions result when an individual's immune system overreacts, or reacts inappropriately, to an encountered antigen. Typically, there is no allergic reaction the first time an individual is exposed to a particular antigen. However, it is the initial response to an antigen that primes the system for subsequent allergic reactions. In particular, the antigen is taken up by antigen presenting cells (APCs; e.g., macrophages and dendritic cells) that degrade the antigen and then display antigen fragments to T cells. T cells, in particular CD4⁺ “helper” T-cells, respond by secreting a collection of cytokines that have effects on other immune system cells. The profile of cytokines secreted by responding CD4⁺ T cells determines whether subsequent exposures to the antigen will induce allergic reactions. Two classes of CD4⁺ T cells (Th1 and Th2) influence the type of immune response that is mounted against an antigen.

Th2 cells can secrete a variety of cytokines and interleukins including IL-4, IL-5, IL-6, IL-10 and IL-13. One effect of IL-4 is to stimulate the maturation of B cells that produce IgE antibodies specific for the antigen. Allergic responses to allergens are characterized by the production of antigen-specific IgE antibodies that are dependent on help from IL-4 secreting CD4⁺ T cells. These antigen-specific IgE antibodies attach to receptors on the surface of mast cells, basophils and eosinophils, where they act as a trigger to initiate a rapid immune response to the next exposure to antigen. When the individual encounters the antigen a second time, the antigen is quickly bound by these surface-associated IgE molecules. Each antigen typically has more than one IgE binding site, so that the surface-bound IgE molecules quickly become crosslinked to one another through their simultaneous (direct or indirect) associations with antigen. Such cross-linking induces mast cell degranulation, resulting in the release of histamines and other substances that trigger allergic reactions. Individuals with high levels of IgE antibodies are known to be particularly prone to allergies.

Current treatments for allergies involve attempts to “vaccinate” a sensitive individual against a particular allergen by periodically injecting or treating the individual with a crude suspension of the raw allergen. The goal, through controlled administration of known amounts of antigen, is to modulate the IgE response mounted in the individual. If the therapy is successful, the individual's IgE response is diminished, or can even disappear. However, the therapy requires several rounds of vaccination, over an extended time period (3-5 years), and very often does not produce the desired results. Moreover, certain individuals suffer anaphylactic reactions to the vaccines, despite their intentional, controlled administration.

Many formulations have been developed to vaccinate individuals against one or more antigens. In their simplest form, these consist of antigen suspended in a saline solution. Over the last two decades, formulations have been developed which have been used to modify the immune response, allow administration of antigen orally or nasally instead of by injection, enhance phagocytosis of the antigen, or provide sustained or intermittent delivery of the antigen in order to induce a particular response. These formulations include liposomal and polymeric formulations.

A search of the literature for formulations designed to deliver allergens for treatment of allergic or autoimmune conditions yields little. The conventional wisdom has been that allergens must be administered in small amounts, by injection, in order to induce tolerance. A recent report of oral administration of a peanut allergen, Arah2, in chitosan microparticles, Roy, et al., Nat. Med. 5(4):387-391 (1999), indicated that oral administeration of the allergen in chitosan nanoparticles could be used to alter IgE-mediated anaphylaxis reactions to the peanut allergen. Arora and Ganal, Asian Pac. J. Allergy Immunol. 16(2-3):87-91 (1998) reported that liposome entrapped allergen can be used to reduce plasma histamine induced by exposure to allergen. Neither of these reports, however, provides a method for treatment.

Autoimmune Diseases

A breakdown of tolerance by an individual's immune system to self antigens can result in autoimmune diseases where the immune system recognizes and attacks cells and tissues of self. The resulting immune response can lead to a variety of debilitating diseases such as insulin-dependent diabetes mellitus (IDDM). Reports in the literature have suggested that modulating the Th1/Th2 responses may modify the course of the disease.

In general, organ-specific autoimmune diseases develop as result of the activation of self reactive Th1-type CD4⁺ T cells. For example, experimental autoimmune encephalomyelitis (EAE) is a model of multiple sclerosis, another autoimmune disease. EAE can be induced in rodents by immunization with myelin basic protein (MBP), proteolipid protein (PLP) or peptides. The immune response in EAE is characteristic of a Th1 type response (for reviews, see Charlton et al. Curr. Opin Immun. 7:793-798, 1995; O'Garra et al. Curr. Opin Immun. 9:872-883, 1997; King et al. Curr. Opin Immun. 9:863-871, 1997).

Studies on the murine model of IDDM provide evidence to suggest that Th1 cells are involved in the development of the disease. Analysis of cytokine production by islet-infiltrating cells demonstrates a correlation between islet destruction and IFN-production, which is an indication of a Th1-type immune response. Additionally, for animal models of rheumatoid arthritis, the addition of IL-12 as an adjuvant has been shown to result in a high incidence of collagen induced arthritis.

However, the relationship between Th1/Th2 responses and autoimmunity is complex. Th2 cells have also been implicated in mediating autoimmune disease. Chronic immunity during host-versus-graft or graft-versus-host responses is mediated by Th2 cells. In addition, MBP-specific Th2 cells, generated in vitro, can stimulate EAE when transferred to immunodeficient rodents (Lafaille et al. J. Exp. Med. 186:307-312, 1997). Therefore, once the relationship between a particular autoimmune disease and the Th1/Th2 response is elucidated, the ability to control Th1/Th2-type response will aid in the treatment of autoimmune related diseases.

It is therefore an object of the present invention to provide formulations and methods for use thereof to reduce allergic reactions to allergens.

It is a further object of the present invention to provide formulations and methods for use thereof to reduce the severity or frequency of the symptoms of asthma.

It is another object of the present invention to provide formulations and methods for use thereof to reduce autoimmune responses.

### SUMMARY OF THE INVENTION

Formulations have been developed for modulating an individual's immune response, especially allergic individuals or individuals who are at risk of developing allergies, so that the severity of the reaction or risk of anaphylactic reaction to the antigens is reduced. In particular, formulations and methods have been developed for delivering antigens to individuals in a manner that substantially reduces contact between the antigen and IgE antibodies displayed on the surfaces of cells involved in mediating allergic responses. The formulations target delivery of antigen to dendritic and other phagocytic APCs, and have improved pharmacokinetics as compared to delivery of the antigens by conventional means. Cells involved in mediating allergic responses include mast cells that are capable of releasing vasoactive substances such as histamines and leukotrienes. By reducing direct and indirect association of antigens with antigen-specific IgE antibodies, the risk of an allergic reaction, possibly anaphylatic shock, is reduced or eliminated.

In the preferred embodiments, the compositions include one or more antigens in a delivery material such as a polymer, either a natural polymer which is targeted to specific tissues or cells or which has been stabilized, or synthetic polymers such as polylactide-co-glycolide copolymers, in the form of particles or a gel, or lipid vesicles or liposomes which are stabilized or targeted to enhance delivery. Preferably, the antigen is surrounded by the encapsulation material. Alternatively or additionally, the antigen is displayed on the surface of the encapsulation material. One result of encapsulating antigen is the reduction in association between allergen and antigen-specific IgE antibodies. In some embodiments, antigens are stabilized or protected from degradation until the antigen can be recognized and endocytized by APCs which are involved in eliciting cellular and humoral immune responses.

In a preferred embodiment, the formulation is designed to deliver antigens to individuals in a manner that promotes a Th1-type mediated immune response and/or in a manner that suppresses a Th2 response. Molecules can be incorporated into or onto the formulations to promote a particular desired response. Substances that promote a Th1-type mediated immune response include cytokines, IL-2, IL-12, IL-18, IFN-gamma, and TNF. Substances that promote a Th2-type mediated immune response include cytokines, IL-4, IL-5, IL-6, IL-10, and IL-13. In still another embodiment, the formulation effects preferential release of the antigen within APCs. In this embodiment, the formulation is stable at physiological pH and degrades at acidic pH levels comparable to those found in the endosomes of APCs.

Preferred antigens or fragments thereof are those that elict allergic reactions in individuals. Particularly preferred antigens are those that may elicit anaphylaxis in individuals, including food antigens, insect venom and rubber-related antigens.

The encapsulated antigen is administered by injection, orally, or topically by application to a mucosal surface (buccal, nasal, pulmonary, rectal). In the preferred embodiment for treatment of allergic individuals, the antigen is administered by injection in an amount and formulation to enhance phagocytosis by APC, while minimizing IgE production and binding and crosslinking of IgE on mast cells, basophils, and other cells having IgE receptors on their surfaces, which could lead to anaphylaxis. In the preferred treatment of individuals with autoimmune diseases associated with a Th1-type immune response, substances that induce Th2-type immune responses are delivered to individuals to downregulate the Th1-type immune responses. For treatment of individuals with autoimmune diseases that are associated with a Th2-type immune response, antigens that induce Th1-type immune responses are delivered to individuals to downregulate the Th2-type immune responses.

### DETAILED DESCRIPTION OF THE INVENTION

Formulations have been developed for modulating an individual's immune response, especially allergic individuals or individuals who are at risk of developing allergies, so that the severity of the reaction or risk of anaphylactic reaction to the antigens is reduced. The same technology can also be used in the treatment of individuals with asthma and autoimmune disorders. In particular, formulations and methods have been developed for delivering antigens to individuals in a manner that substantially reduces contact between the antigen and IgE antibodies displayed on the surfaces of cells involved in mediating allergic responses. The formulations target delivery of antigen to dendritic and other phagocytic APCs, and have improved pharmacokinetics as compared to delivery of the antigens by conventional means. The formulation results in the APCs taking up the antigen prior to exposure or release of the allergen where it can bind to and crosslink IgE receptors on the cell surfaces. By reducing direct and indirect association of antigens with antigen-specific IgE antibodies, the risk of an allergic reaction, possibly anaphylatic shock, is reduced or eliminated.

In the preferred embodiments, the compositions include one or more antigens in a delivery material such as a polymer, either a natural polymer which is stabilized or targeted to specific tissues or cells, synthetic polymers such as polylactide-co-glycolide copolymers, in the form of particles or a gel, or lipid vesicles or liposomes which are stabilized or targeted to enhance delivery. Preferably, the antigen is surrounded by the encapsulation material. Alternatively or additionally, the antigen is displayed on the surface of the encapsulation material. One result of encapsulating antigen is the reduction in association with antigen-specific IgE antibodies. In some embodiments, antigens are stabilized or protected from degradation until the antigen can be recognized and endocytized by APCs which are involved in elicting cellular and humoral immune responses. Adjuvants can also be incorporated into the formulation or materials which act as adjuvants used to form the encapsulating matrix.

The formulations are used to induce tolerance, which is essentially the same response induced with allergy shots typically administered over a period of three to five years, but in a shorter time frame and with a lower risk of anaphylaxis.

Definitions

“Activated cells”: An “activated” or “sensitized” cell is a cell such as a mast cell, basophil or other cell that has surface-bound anti-antigen IgE molecules. The term is antigen specific. That is, at any given time, a particular cell will be “activated” against certain antigens (those that are recognized by the IgE on its surface) but will not be activated against other antigens.

“Allergen”: An “allergen” is an antigen that (i) elicits an IgE response in an individual; and/or (ii) elicits an asthmatic reaction (e.g., chronic airway inflammation characterized by eosinophilia, airway hyperresponsiveness, and excess mucus production), whether or not such a reaction includes a detectable IgE response).

“Allergic individual”: “Allergic individual” refers to an individual with sensitivities to particular allergens as exhibited by the production of IgE sufficient to cause a measurable clinical response. Such an individual has a reaction to a relatively innocuous antigen, causing a harmful immune response and/or tissue damage. Symptoms of allergy may consist of exaggerated or pathological reaction (e.g., sneezing, respiratory distress, itching, or skin rashes) to substances, situations or physical states that are without comparable effect on the average individual.

“Allergy”: “Allergy” refers to a state of hypersensitivity induced by exposure to a particular antigen resulting in harmful immunologic reactions on subsequent exposures. In particular, “allergy” includes, but is not limited to, hypersensitivity to an environmental antigen (atopic allergy or contact dermatitis) or to drug allergy. Harmful immunologic reactions include but are not limited to anaphylaxis, asthma, shortness of breath, rash, wheezing, and hypotension.

“Anaphylactic antigen”: An “anaphylactic antigen” is an antigen that is recognized to present a risk of anaphylactic reaction in allergic individuals when encountered in its natural state, under natural conditions. For example, for the purposes of the present invention, pollens and animal danders or excretions (e.g., saliva, urine) are not considered to be anaphylactic antigens. On the other hand, food antigens, fish, crustaceans, tree nuts, insect antigens, and latex are generally considered to be anaphylactic antigens.

“Anaphylaxis” or “anaphylactic reaction”, as used herein, refers to an immune response characterized by inflammatory reactions resulting from a combination of a soluble antigen with IgE bound to a mast cell that leads to degranulation of the mast cell and release of histamine and histamine like substances, causing localised or global immune reponses. The result is an acute allergic reaction with shortness of breath, rash, wheezing, hypotension.

“Antigen”: An “antigen”: is (i) any compound that elicits an immune response; and/or (ii) any compound that binds to a T cell receptor or to an antibody produced by a B-cell. Preferred antigens are protein antigens, but antigens need not be proteins.

“Asthmatic individual”: refers to an individual who experiences asthmatic symptoms (e.g., chronic airway inflammation characterized by eosinophilia, airway hyperresponsiveness, and excess mucus production) upon inhalation of a particular substance or antigen. Asthmatic individuals, in contrast to allergic individuals, do not necessarily exhibit a detectable production of IgE.

“T cell stimulation”: Certain antigen fragments stimulate Th1 helper T cells preferentially as compared with their ability to stimulate Th2 helper cells.

Compositions and Methods for Encapsulation and Targeting of Antigens

Encapsulation of antigens may substantially reduce direct and indirect association of antigens with antigen-specific IgE antibodies bound to surface of cells such as mast cells. As a result, allergic reactions caused by the release of histamines, leukotrienes and other vasodilators by mast cells due to activation by IgE crosslinking can be substantially reduced or avoided. Alternatively or additionally, encapsulation of antigens may stabilize antigens and prevent premature degradation of antigens before endocytosis by antigen-presenting cells (i.e., preventing release or degradation of an amount of antigen which could elicit an anaphylaxis reaction). Encapsulation also allows for the co-presentation of antigen and adjuvant and targeting of the cells, for example, to induce a TH1 or TH2 response, using substances which promote a Th1-type mediated immune response and/or suppress a Th2 response. Substances that promote a Th1-type mediated immune response include the cytokines IL-2, IL-12, IL-18, IFN-gamma, and TNF. Substances that promote a Th2-type mediated immune response include the cytokines IL-4, IL-5, IL-6, IL-10, and IL-13. Antagonists or inhibitors of these cytokines can therefore be used to suppress a T helper cell 2 response.

A number of encapsulation technologies are known to those skilled in the art. These include formation of particles, which can include microparticles and nanoparticles, microspheres, and microcapsules, liposomes and phospholipid vesicles, slabs, disks, beads, tablets, films, and gels.

The antigen can be encapsulated or entrapped within the carrier, depending on the composition and method of encapsulation. In some cases antigen may be entrapped in the core of the carrier, while in others antigen may be incorporated into the carrier.

The material used to encapsulate the antigen can be biodegradable or non-biodegradable. Most biodegradable materials degrade either upon exposure to enzymes present in the body or hydrolytically. Some materials release encapsulated antigen by diffusion, degradation or a combination of diffusion and degradation. Alternatively, some materials disaggregate upon exposure to a change in pH or temperature, to release entrapped antigen. Coatings can be used to alter release, for example, an enteric coating can be used to protect the carrier and entrapped antigen when administered orally, so that it is released intact upon reaching the small intestine. Polyalkyleneglycol moieties on the surface of the carrier can be used to avoid uptake by the reticuloendothelial system (RES), when desirable.

Preferably, encapsulation compositions allow release of antigens from within macrophages and dendritic cells by degrading at a pH level comparable to cytoplasmic and/or endosomal pH levels (typically between 4-6, more usually, approximately pH=5) while maintaining stability at physiological pH levels (typically pH 7). See, for example, Park et al. J. Control. Rel. 33:211-222 (1995); Vert et al. J. of Controlled Release. 16:15-26 (1991); and Witschi and Doelker. J. of Controlled Release. 51:327-341 (1998).

Polymeric Materials for Encapsulation of Antigen

Rapidly bioerodible polymers such as poly[lactide-co-glycolide], polyanhydrides, and polyorthoesters, whose carboxylic groups are exposed on the external surface as their smooth surface erodes, are excellent candidates for bioadhesive drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, chitosan, casein, gelatin, gluten, serum albumin, or collagen, and polysaccharides, such as celluloses (including modified celluloses such as alkyl celluloses, hydroxyalkyl celluloses, cellulose ethers, cellulose esters, and nitrocelluloses, for example, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt), dextrans, polyhyaluronic acid and alginic acid. Representative synthetic polymers include polyhydroxyacids, polyphosphazines, poly(vinyl alcohols), polyamides, polycarbonates, polyalkylenes, polyacrylamides (including polymers of acrylic and methacrylic esters and copolymers thereof, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene), polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Preferred bioerodible polymers include polylactides, polyglycolides and copolymers thereof, poly(ethylene terephthalate), poly(butyric acid), poly(valeric acid), poly(lactide-co-caprolactone), poly[lactide-co-glycolide], polyanhydrides, polyorthoesters, blends and copolymers thereof.

Suitable molecular weights for polymers can be determined by a person of ordinary skill in the art taking into consideration factors such as the desired polymer degradation rate, physical properties such as mechanical strength, and rate of dissolution of polymer in solvent. Typically, an acceptable range of molecular weights is within about 2,000 Daltons to about 2,000,000 Daltons. Most preferably, the polymer is a biodegradable polymer or copolymer. A particularly preferred polymer is a poly(lactide-co-glycolide) (hereinafter “PLGA”) with a molecular weight of about 5,000 Daltons to about 70,000 Daltons. PLGA compositions may be altered to affect the rate of release of antigens. One of ordinary skill in the polymer art will recognize that factors affecting degradation including the ratio of lactide to glycolide, the polymer molecular weight and stereochemistry of lactic acid and glycolic acid may be altered. See Cohen, Alonso, and Langer. Int. J. of Tech. Assess. in Health Care. 10:121-130 (1994) for a review. PLGA compositions may also be altered to affect the rate of release according to the environmental factors such as the pH level.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

Formation of Polymeric Microparticles, Microspheres, or Microcapsules.

Several methods for preparing microparticles (which will be understood to encompass microcapsules, microspheres and nanoparticles, with the understanding that the actual conformation of the particles will be determined by the chemical composition of the particle and method of manufacture) are well known in the art. As used herein, the term “microspheres” includes microparticles and microcapsules (having a core of a different material than the outer wall), having a diameter in the nanometer range up to 5000 microns. Microparticles of less then ten microns and more preferably less than five microns are preferred for uptake by phagocytic cells. The microparticles may consist entirely of polymer or have only an outer coating of polymer. Microparticles may also consist of non-polymeric materials, such as liposomes.

Examples of these processes include single and double emulsion solvent evaporation, spray drying, solvent extraction, solvent evaporation, phase separation, simple and complex coacervation, and interfacial polymerization. Methods developed for making microparticles for drug delivery are described in the literature, for example, in Doubrow, ed., “Microcapsules and Nanoparticles in Medicine and Pharmacy” (CRC Press, Boca Raton 1992) and Benita, ed., “Microencapsulation: Methods and Industrial Applications” (Marcel Dekker, Inc., New York 1996).

Emulsion Based Methods

Emulsion-based processes usually begin with the preparation of two separate phases: a first phase, which generally consists of a dispersion or solution of an active agent in a solution of polymer dissolved in a first solvent, and a second phase, which generally consists of a solution of surfactant and a second solvent that is at least partially immiscible with the dispersed phase. After the first and second phases are prepared, they are combined using dynamic or static mixing to form an emulsion, in which microdroplets of the first phase are dispersed in the second, or continuous, phase. The microdroplets then are hardened to form polymeric microparticles that contain the active agent. The hardening step is carried out by removal of the first solvent from the microdroplets, generally by either an extraction or evaporation process.

Solvent Extraction or Removal

In this method, the drug is dispersed or dissolved in a solution of the selected polymer in a volatile organic solvent like methylene chloride. This mixture is suspended by stirring in an organic oil (such as silicon oil) to form an emulsion.

Several U.S. patents describe solvent removal by extraction. For example, U.S. Pat. No. 5,643,605 to Cleland et al. discloses an encapsulation process in which the emulsion is transferred to a hardening bath (i.e. extraction medium) and gently mixed for about 1 to 24 hours to extract the polymer solvent. U.S. Pat. No. 5,407,609 to Tice et al. teaches transferring the emulsion to a volume of extraction medium that is preferably ten or more times the volume required to dissolve all of the solvent in the microdroplets, so that greater than 20-30% of the solvent is immediately removed. U.S. Pat. No. 5,654,008 to Herbert et al. similarly discloses a process in which the volume of quench liquid, or extraction medium, should be on the order of ten times the saturated volume.

Unlike solvent evaporation, this method can be used to make microspheres from polymers with high melting points and different molecular weights. Microspheres that range in diameter to between 1-300 microns can be obtained by this procedure. The external morphology of spheres produced with this technique is highly dependent on the type of polymer used.

Solvent Evaporation

Evaporation is another approach known in the art for solvent removal. For example, U.S. Pat. No. 3,891,570 to Fukushima et al. and U.S. Pat. No. 4,384,975 to Fong teach solvent removal by evaporating an organic solvent from an emulsion, preferably under reduced pressure or vacuum. See also solvent evaporation, as described by E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984).

Generally, the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid microspheres. The solution is loaded with antigen and suspended in vigorously stirred distilled water containing 1% (w/v) surfactant such as poly(vinyl alcohol). The organic solvent evaporates from the polymer, and the resulting microspheres are washed with water and dried overnight in a lyophilizer. Microspheres with different sizes (1-1000 microns) and morphologies can be obtained by this method.

The foregoing methods may also be combined. For example, U.S. Pat. No. 4,389,330 to Tice et al. (“Tice '330”). Tice '330 describes an emulsion-based method for making drug-loaded polymeric microspheres that uses a two-step solvent removal process: evaporation followed by extraction. The evaporation step is conducted by application of heat, reduced pressure, or a combination of both, to remove between 10 and 90% of the solvent.

Hot-melt Encapsulation

Hot-melt encapsulation is typically used only with polymers having a low melting point, for example, polyanhydrides, and is performed for example as described by E. Mathiowitz, et al., Reactive Polymers, 6, 275 (1987). In this method, the polymer is first melted and then mixed with the solid particles of dye or drug that have been sieved to less than 50 microns. The mixture is suspended in a non-miscible solvent (like silicon oil), and, with continuous stirring, heated to 5 C above the melting point of the polymer. Once the emulsion is stabilized, it is cooled until the polymer particles solidify. The resulting microspheres are washed by decantation with petroleum ether to give a free-flowing powder. Microspheres with sizes between one to 1000 microns are obtained with this method. The external surfaces of spheres prepared with this technique are usually smooth and dense. This procedure is used to prepare microspheres made of polyesters and polyanhydrides. However, this method is limited to polymers with molecular weights between 1000-50,000.

Spray Drying

Spray drying is another common technique for making particles for drug delivery. In brief, a solution or suspension of antigen and polymer is made, then atomized under conditions removing the polymer solvent. For example, the polymer is dissolved in methylene chloride (0.04 g/mL). A known amount of the active drug is suspended (insoluble drugs) or co-dissolved (soluble drugs) in the polymer solution. The solution or the dispersion is then spray-dried. Typical process parameters for a mini-spray drier (Buchi) are as follows: polymer concentration 0.04 g/mL, inlet temperature −24 C, outlet temperature 13-15 C, aspirator setting=15, pump setting=10 mL/minute, spray flow=600 Nl/hr, and nozzle diameter 0.5 mm. Microspheres ranging between 1-10 microns are obtained with a morphology which depends on the type of polymer used. This method is primarily used for preparing microspheres having a particle size not in excess of 10.

Hydrogel Microparticles

Microspheres made of gel-type polymers, such as alginate, chitosan, alginate/polyethylenimide (PEI) and carboxymethyl cellulose (CMC), are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microspheres are left to incubate in the bath for twenty to thirty minutes in order to allow sufficient time for gelation to occur. Microsphere particle size is controlled by using various size extruders or varying either the nitrogen gas or polymer solution flow rates.

Other hydrogel microparticle compositions comprise a reversibly gelling polymeric network. Such networks comprise a responsive polymer component capable of aggregation in response to an environmental stimulus (see Ron et al. WO 98/06438 incorporated herein by reference). Preferably, the polymer network is a reversably thermally viscosifying polymer network. The polymer network includes at least one responsive polymer component which is capable of aggregation insolution in response to an environmental stimulus and also includes at least one structural component which exhibit self-repulsive interactions under conditions of use. The responsive component is randomly bonded to the structural component. The polymer network is characterized by its ability to viscosify in response to environmental stimuli.

Preferably, the polymer network contains about 0.01-20 percent by weight of each of the response polymer and the structural polymer. Particularly preferred polymer network compositions range from a ratio of about 1:10 to about 10:1 response polymer:structural polymer. Also preferred are polymer network gel compositions which exhibit a reversible gelation at body temperature (approximately 37 C±5 C). For particularly preferred polymers, see Ron et al. WO 98/06438.

Materials that Release as a Function of pH

Some polymeric materials aggregate under certain conditions to encapsulate or incorporate antigen within the microparticle, then release upon exposure to a stimulus such as a change in pH or temperature. An example of microparticles that release as a function of a change in pH include the diketopiperazine particles described in U.S. Pat. No. 5,352,461 issued Oct. 4, 1994 “Self-Assembling Diketopiperazine Drug Delivery System” to Steiner, et al., and the proteinoid formulations described in U.S. Pat. No. Reissue 35,862 issued Jul. 28, 1998, for “Delivery Systems for Pharmacological agents Encapsulated with Proteinoids”.

Liposomal Formulations

In some embodiments, it may be desirable to deliver antigen in a liposomal formulation. In some cases, liposomes can be used to enhance delivery by phagocytosis. Liposomes have been known for many years. These are formed by emulsifying amphipathic molecules, such as phospholipids, in an aqueous solution, where the molecules form spheres, having the hydrophobic ends of the molecules orient towards each other and the hydrophilic ends pointing outwards, forming one or more bilayers. Liposomes may be unilamellar vesicles (possessing a single bilayer membrane) or multilamellar vesicles (onion-like structures characterized by multiple membrane bilayers, each separated from the next by an aqueous layer). Examplary liposomes are described in U.S. Pat. No. 5,916,588.

Preparation of liposomes is described by Bangham, et al., J. Mol. Biol., 12:238-252 (1965). Phospholipids are dissolved in an organic solvent which is then evaporated to dryness leaving a phospholipid film on the reaction vessel. Next, an appropriate amount of aqueous phase is added, the mixture is allowed to “swell,” and the resulting liposomes which consist of multilamellar vesicles (MLVs) are dispersed by mechanical means. This technique provides the basis for the development of the small sonicated unilamellar vesicles described by Papahadjopoulos et al. Biochim. Biophys. Acta., 135:624-638 1968), and large unilamellar vesicles. Small unilamellar vesicles have a diameter of about 100 nm or less. Unilamellar vesicles with a defined size can be prepared by extrusion through a polycarbonate filter, using standard techniques, for example, as described in PCT Application No. WO 87/00238 by Cullis, et al.

Since liposomes typically have very short half-lives in vivo as well as during storage, various methods have been used to enhance stability. U.S. Pat. No. 5,820,880 issued Oct. 13, 1998 to Alving describes liposomal formulations stabilized with a non-ionic detergent polymerized liposomes. Polymerized liposomes contain covalent and/or ionic crosslinks between the amphipathic molecules to stabilize the structures. For example, U.S. Pat. No. 5,762,904 issued Jun. 9, 1998 to Okada, et al., describes polymerized liposomes, methods of preparing the polymerized liposomes and incorporating biologically active substances within the polymerized liposomes, and methods of administering polymerized liposomes containing a biologically active substance to be delivered to a patient. The polymerized liposomes are prepared by polymerizing double bond-containing liposomes. The polymerization can be initiated with a source of radiation and/or a free radical initiator. Biologically active substances can be incorporated into both the hydrophilic and hydrophobic layers of the liposomes, either during or after polymerization. The polymerized liposomes have the additional advantage that they can be administered orally to a patient.

Liposomal formulations for administration of vaccines including an adjuvant are described in U.S. Pat. No. 5,919,480 issued Jul. 6, 1999 to Keddar, et al. U.S. Pat. No. 5,709,879 issued Jan. 20, 1998, to Barchfeld, et al. describes a vaccine composition comprising an antigenic substance in association with a liposome and an oil-in-water emulsion comprising a muramyl peptide, a metabolizable oil, and optionally an additional emulsifying agent. The two components of the adjuvant (i.e., the liposome/antigen component and the emulsion component) act together to produce high levels of immune response.

Formulations to Promote a Particular T cell response or to Target or Enhance Binding In a preferred embodiment, liposomal formulations containing encapsulated antigens that induce Th1 immune responses or downregulate Th2 responses are used. Particularly preferred liposomal encapsulation compositions are stable at physiological pH and slowly degrade at acidic pH levels characteristic of endosomes (approximately pH 5). Numerous studies have been conducted which examine the stability of liposomal encapsulation compositions and the ability of acidic pH to destabilize the liposome for drug release (see Kono et al. Biochim. Biophys. Acta. 1325:143-154 and references within). Lee et al. Bioscience Reports. 18:69-78 (1998) demonstrated that pH triggered release of liposome-encapsulated drugs occurs in the endosome following receptor-mediated endocytosis. Liposome encapsulated antigens have been shown to elict Th1 immune responses for therapeutic cancer vaccines by Guan et al. Bioconjugate Chem. 9:451-458 (1998). These researchers demonstrated that physical association of a peptide antigen with liposomes either through surface-exposure or through encapsulation resulted in a strong Th1-type immune response. Shahum and Therien, Int. J. Immunopharmac. 17:9-20 (1995) also showed that liposomal-associated antigens induce Th1-type immune responses. Their study indicated that both encapsulated and surface-linked liposomal antigens induce a Th1 type immune response but that surface linkage favors a more rapid maturation of the induction and a much more intense immunity help induction. However, this must be moderated with the need to present antigen so that it is not available to crosslink IgE on the surface of cells such as mast cells.

A similar presentation of antigen and molecules on polymeric microparticles can also be used to induce a Th1 type immune response. Methods for encapsulation which incorporate the antigen into and onto the surface of the microparticles are preferably used to form the microparticles. Alternatively, the antigen can be coupled directly to the polymer for incorporation at the time of formation of the microparticle or coupled to the surface of the microparticle after formation.

In some embodiments, it may be desirable to derivatize or modify the carrier and/or the antigen to enhance delivery to APCs such as dendritic cells, to promote T helper cell 1 responses, or to enhance adhesion to mucosal surfaces, such as the lining of the gut following oral administration, or the lungs or nasal passageways when the encapsulated antigen is administered by aerosol or inhalation, to increase tolerance. Ligands may be attached to the polymers to target the delivery of the carrier or to enhance adhesion to a particular tissue or cell type. Antibodies and antibody fragments are examples of well known tissue or target specific ligands.

Dendritic cells are characterized by certain markers, some of which are expressed at different levels as a function of the maturity of the cells or as a function of the tissue source of the dendritic cells (lung versus skin) (see, for example, Cochand, et al., Am. J. Respir. Cell Mol. Biol. 21(5):547-554 (1999)). Examples of these ligands include CD83, major histocompatibility complex (MHC) Class II molecules, CCR1 and CCR5, and costimulatory molecules CD40, CD80, and CD86. Fractalkine, a CX3C chemokine, is expressed by dendrictic cells and up-regulated upon dendritic cell maturation (Papadopoulos, et al., Eur. J. Immunol. 29(8):2551-2559 (1999). CMRF-56 and CMRF-44 are cell surface antigens whose expression is restricted to human dendritic cells (Hock, et al., Tissue Antigens 53(4 Pt 1):320-334. Alternatively, encapsulated antigens could be targeted to APCs especially dendritic cells or macrophages via association with a ligand that interacts with an uptake receptor such as the mannose receptor or an Fc receptor. Encapsulated antigens could be targeted to other APCs via association with a ligand that interacts with the complement receptor. Antigens or encapsulated antigens could be specifically directed to dendritic cells by association with DEC205, a mannose-like receptor that is specific for these cells.

Mucosal adhesion can be enhanced by modifying the polymers by increasing the number of carboxylic groups accessible during biodegradation, or on the polymer surface. The polymers can also be modified by binding amino groups to the polymer. The polymers can also be modified using any of a number of different coupling chemistries that covalently attach ligand molecules with bioadhesive properties to the surface-exposed molecules of the polymeric microspheres. The attachment of any positively charged ligand, such as polyethyleneimine or polylysine, can improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most microspheres with the appropriate chemistry, such as CDI, and be expected to influence the binding of the polymeric material to the gut. For example, polyclonal antibodies raised against components of mucin or else intact mucin, when covalently coupled to microspheres, would provide for increased bioadhesion. Similarly, antibodies directed against specific cell surface receptors exposed on the lumenal surface of the intestinal tract will increase the residence time of beads, when coupled to microspheres using the appropriate chemistry. The ligand affinity need not be based only on electrostatic charge, but other useful physical parameters such as solubility in mucin or else specific affinity to carbohydrate groups. Useful ligands include sialic acid, neuraminic acid, n-acetyl-neuraminic acid, n-glycolylneuraminic acid, 4-acetyl-n-acetylneuraminic acid, diacetyl-nacetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface. Polyamino acids containing extra pendant carboxylic acid side groups, e.g., polyaspartic acid and polyglutamic acid, will also provide a useful means of increasing bioadhesiveness.

Ligands can be incorporated within the materials forming the carrier by physical intermixing, or through chemical coupling to molecules forming the carriers. One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

Either of these protocols can be used to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method is useful for attaching sugars or other hydroxylic compounds to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Antigen

In general, any antigen may be encapsulated and delivered. Antigens can be organic or inorganic molecules including many chemicals and drugs, proteins or peptides, polysaccharides, nucleic acids, glycoproteins, which may be immunoreactive alone or in combination with or conjugated to a carrier. Antigen may be in purified or isolated form, or present in extracts or purification fractions of bacteria, viruses, protozoa, plants or animals. Any antigen that potentially crosslinks IgE antibodes may be used. Preferred antigens are antigens that may induce anaphylaxis, such as some protein allergens found in food (peanut, milk, egg, wheat), insect venom, fish, crustaceans, tree nuts, drugs (such as penicillin), and latex rubber proteins. Non-limiting examples of protein allergens found in food include proteins found in nuts, seafood, fruit (e.g. plums, peaches, nectarines; Ann Allergy Asthma Immunol 7(6):504-8 (1996); cherries, Allergy 51(10):756-7 (1996)), soy and dairy products. Some protein allergens found in nuts are related to legume allergies and may be used instead of the legume proteins (e.g. peanuts, soybeans, lentils; Ann Allergy Asthma Immunol 77(6):480-2 (1996). Also, protein antigens found in pollen-related food allergies may be used (e.g. birch pollen related to apple allergies). Other protein allergens found in foods include those found in young garlic (Allergy 54(6):626-9 (1999), and for children allergic to house dust mites, allergens found in snails (Arch Pediatr 4(8):767-9 (1997)). Protein allergens in wheat are known to cause exercise-induced allergies (J Allergy Clin Immunol May 1999; 103(5 Pt 1):912-7). Non-limiting examples of proteins in insect venom that cause anaphylaxis include phospholipase A found in bee venom (Weber et al. Allergy 42:464-470.). Collections of more than one antigen can be used, so that immune responses to multiple antigens may be modulated simultaneously.

Autoantigens or protein eliciting antibodies cross-reactive with autoantigens can also be used. Most autoimmune disorders such as systemic lupus erythematosus (“SLE”) are believed to be caused initially by an infection with a virus such as the Epstein-Barr virus. The body responds by producing antibodies against the virus, which then react not only against the antibody-eliciting antigen, but also the individual's own proteins, such as the Ro/SSA and La antigens, and even nucleic acids such as DNA. These antibodies are referred to as autoantibodies, and cause diseases such as rheumatoid arthritis, multiple sclerosis, Sjrogen's Syndrome, as well as SLE.

The antigen can also be provided as a nucleic acid molecule which is expressed upon delivery to yield a protein allergen. The nucleotide molecule can be provided as naked DNA, in a plasmid or in a viral vector such as an adeno-associated adenoviral vector. Techniques for generating nucleic acids including an expressible gene, and for introducing such nucleic acids into an expression system in which any protein encoded by the expressible gene will be produced, are well established in the art (see, for example, Sambrook et al., Molecular Cloning: A Laboratory Manual, 2nd Ed., Cold Spring Harbor Laboratory Press, Cold Spring Harbor, N.Y., 1989).

Where the antigen is a peptide, it may be generated, for example, by proteolytic cleavage of isolated proteins. Any of a variety of cleavage agents may be utilized including, but not limited to, pepsin, cyanogen bromide, trypsin, and chymotrypsin. Alternatively, peptides may be chemically synthesized, preferably on an automated synthesizer such as is available in the art (see, for example, Stewart et al., Solid Phase Peptide Synthesis, 2d. Ed., Pierce Chemical Co., 1984). Also, recombinant techniques may be employed to create a nucleic acid encoding the peptide of interest, and to express that peptide under desired conditions (e.g., in a host cell or an in vitro expression system from which it can readily be purified). Preferred embodiments using peptide antigens employ collections of peptides representing fragments of a protein antigen. In certain particularly preferred embodiments, substantially all of the structural elements of the protein antigen with the exception of one of more IgE-binding sites is represented in the peptide collection.

The amount of antigen to be employed in any particular composition or application will depend on the nature of the particular antigen and of the application for which it is being used, as will readily be appreciated by those of ordinary skill in the art. In general, larger amounts of antigen are useful for inducing Th1 responses, smaller amounts for inducing Th2 responses.

Adjuvants

Immunologic adjuvants are agents that enhance specific immune responses to vaccines. Formulation of vaccines with potent adjuvants is desirable for improving the performance of vaccines composed of antigens. Adjuvants may have diverse mechanisms of action and should be selected for use based on the route of administration and the type of immune response (antibody, cell-mediated, or mucosal immunity) that is desired for a particular vaccine (see Vogel. “Adjuvants in HIV Vaccine Research.” available at the following Internet address: www.niaid.nih.gov/daids/vaccine/pdf/adjuvants.pdf.

Some polymers are also adjuvants. For example, the polyphosphazenes described in U.S. Pat. No. 5,500,161 to Andriavnov, et al. These can be used not only to encapsulate the antigen but also to enhance the immune response to the antigen.

The cytokine(s) or inducing agent(s) to be administered is/are selected to reduce production of a Th1 or Th2 response, depending on the particular application involved, as discussed above. One preferred method of reducing a Th1 or Th2 response is through induction of the alternative response. Cytokines that, when expressed during antigen presentation to a T cell, induce a Th1 response in T cells (i.e., “Th1 stimulating cytokines”) include IL-12, IL-2, I-18, IL-1 or fragments thereof IFN, and/or IFN; Th2 stimulating cytokines include IL-4. Inducing agents that prompt the expression of Th1 stimulating cytokines include factors such as LPS; monophosphoryl lipid A (MPLA) from gram negative bacterial lipopolysaccharides (Richards et al. Infect Immun 1998. 66(6):2859-65), CD40, CD40 ligand, oligonucleotides containing CpG motifs, TNF, and microbial extracts such as preparations of Staphylococcus aureus, heat killed Listeria, and modified cholera toxin, etc.; inducing agents that prompt the expression of Th2 stimulating cytokines include agents that induce IL-4 expression by T cells or other cells, as well as agents that suppress IL-12 expression.

Cytokines or inducing agents may be provided as impure preparations (e.g., isolates of cells expressing a cytokine gene, either endogenous or exogenous to the cell), but are preferably provided in purified form. Purified preparations are preferably at least about 90% pure, more preferably at least about 95% pure, and most preferably at least about 99% pure. Alternatively, genes encoding the cytokines or inducing agents may be provided, so that gene expression results in cytokine or inducing agent production either in the individual being treated or in another expression system (e.g., an in vitro transcription/translation system or a host cell) from which expressed cytokine or inducing agent can be obtained for administration to the individual.

Where both cytokine/inducing agent and antigen are to be delivered to an individual, they may be provided together or separately. For example, both compounds may be associated by means of a common encapsulation device or by means of physical association such as covalent linkage, hydrogen bonding, hydrophobic interaction, van der Waals interaction, etc. In certain preferred embodiments in which both compounds are provided together, genes encoding both are provided. For example, genes for both may be provided as part of the same nucleic acid molecule. In some embodiments, this nucleic acid molecule may be prepared so that both factors are expressed from a single gene, as a fusion protein in which the cytokine or inducing agent and the antigen are covalently linked to one another via a peptide bond. Alternatively or additionally, the genes may be linked to the same or equivalent control sequences, so that both genes become expressed within the individual in response to the same stimuli. A wide variety of different control sequences, active in different host cells under different conditions is available in the art. Any such control sequences, including constitutive control sequences, inducible control sequences, and repressible control sequences, may be used in accordance with the present invention, though inducible or repressible sequences are particularly preferred for applications in which additional control over the timing of gene expression is desired.

Coordinate control is particularly desirable where one or more of the cytokines, inducing agents, or antigens being employed is a heterodimeric compound (e.g., IL-12). In such cases, it will generally be desirable to express both dimer components at comparable levels, preferably under control of the same regulatory elements. Also, fusions may be made with one or both dimer components.

It will be appreciated by those of ordinary skill in the art that administration of cytokine and/or antigen may optionally be combined with the administration of any other desired immune system modulatory factor such as, for example, an adjuvant or other immunomodulatory compound. A compendium of vaccine adjuvants, Vogel et al. “A Compendium of Vaccine Adjuvants and Excipients.” 2nd Ed., is available at the following Internet address: http://www.niaid.nih.gov/daids/vaccine/pdf/compendium.pdf). Particularly preferred are ones that induce IL-12 production, including microbial extracts such as fixed Staphylococcus aureus, Streptococcal preparations, Mycobacterium tuberculosis, lipopolysaccharide (LPS), monophosphoryl lipid A (MPLA) from gram negative bacterial lipopolysaccharides (Richards et al. Infect Immun June 1998;66(6):2859-65), listeria monocytogenes, toxoplasma gondii, leishmania major.

Methods of Administration

Formulations can be delivered to a patient by enteral, parenteral, topical (including nasal, pulmonary or other mucosal route), oral or local administration. The compositions are preferably injected in an amount effective to minimize IgE production and/or IgE mediated responses.

The present invention will be further understood by reference to the following non-limiting example.

### EXAMPLE 1

### Preparation of an Encapsulated Allergen Formulation

Various synthetic, biodegradable polymeric microsphere formulations were prepared containing peanut allergen. These were tested to determine the location of the peanut proteins used to load these microspheres. Formulation #179-47-01, D,L-polylactide-co-glycolide (“PLG”) 75:25 containing an acid end group, (0.1 wt % loaded with allergen) had the lowest amount (<20 ng) of peanut protein detected on the outside of the microsphere and the best range of peanut protein allergens contained within the microspheres (having molecular weights ranging from 15 kDa to 70 kDa). In vivo experiments in peanut sensitized mice are currently underway to determine suitability for use in a clinical trial of human subjects.

Modifications and variations of the methods and compositions described herein are intended to be within the scope of the following claims.

We claim:

1. A composition for delivery of an antigen to antigen presenting or phagocytic cells comprising
a synthetic polymeric carrier, targeted polymeric carrier or crosslinked or targeted lipid carrier which is stable until phagocytized or endocytosed by the cells, encompassing an antigen,
wherein the composition elicits less of an IgE mediated immune response than administration of the antigen in the absence of the carrier.

2. The composition of claim 1 wherein the cells are dendritic cells or macrophages.

3. The composition of claim 1 wherein the carrier is a natural polymeric material targeted using antibody or antibody fragment or ligand for molecules specific to or preferentially expressed on the surface of the antigen presenting or phagocytic cells.

4. The composition of claim 1 wherein the carrier is a crosslinked or stabilized liposome or lipid vesicle having antigen encapsulated therein.

5. The composition of claim 4 targeted using antibody or antibody fragment or ligand for molecules specific to or preferentially expressed on the surface of the antigen presenting or phagocytic cells.

6. The composition of claim 1 wherein the carrier is formed of a synthetic polymeric carrier.

7. The composition of claim 6 wherein the carrier is biodegradable by enzymes or hydrolysis.

8. The composition of claim 1 wherein the antigen is an allergen which can crosslink IgE and induce anaphylaxis.

9. The composition of claim 8 wherein the antigen is selected from the group consisting of egg proteins, soybean proteins, peanut proteins, latex rubber proteins, milk proteins, wheat proteins, fish, crustaceans, tree nuts, and insect venom proteins.

10. The composition of claim 1 wherein the antigen is an autoantigen or protein eliciting antibodies cross-reactive with autoantigens.

11. The composition of claim 1 wherein the antigen crosslinks IgE receptors.

12. The composition of claim 1 wherein the antigen is completely encapsulated within the carrier and no antigen is presented on the surface of the carrier.

13. The composition of claim 1 wherein the carrier releases the antigen in response to a change in pH.

14. The composition of claim 13 wherein the carrier releases antigen in response to low pH.

15. The composition of claim 1 wherein the composition activates a T cell helper 1 response.

16. The composition of claim 1 further comprising a pharmaceutically acceptable carrier for administration by injection.

17. The composition of claim 1 further comprising a pharmaceutically acceptable carrier for local or topical administration to a mucosal surface.

18. The composition of claim 1 wherein the composition is formulated to induce a T helper cell 1 response or suppress a T helper cell 2 response.

19. The composition of claim 18 wherein the composition induces a T helper cell 1 response and comprises a cytokine selected from the group consisting of IL-2, IL-12, IL-18, IFN-gamma, and TNF.

20. The composition of claim 18 wherein the composition suppresses a T helper 2 response and comprises an antagonist or inhibitor of a cytokine selected from the group consisting of IL-4, IL-5, IL-6, IL-10, and IL-13.

21. The composition of claim 1 comprising an adjuvant or wherein the carrier is formed of materials acting as an adjuvant.

22. A method for inducing an immune response to an antigen comprising administering to an individual in need thereof a composition delivering an antigen to antigen presenting or phagocytic cells comprising
a synthetic polymeric carrier, targeted polymeric carrier or crosslinked or targeted lipid carrier which is stable until phagocytized or endocytosed by the cells, encompassing an antigen,
wherein the composition elicits less of an IgE mediated immune response than administration of the antigen in the absence of the carrier.

23. The method of claim 22 wherein the antigen presenting or phagocytic cells are dendritic cells or macrophages.

24. The method of claim 22 wherein the carrier is formed of a natural polymeric material and is targeted to antigen presenting or phagocytic cells using antibody or antibody fragment or ligand for molecules specific to or preferentially expressed on the surface of the antigen presenting or phagocytic cells.

25. The method of claim 22 wherein the carrier is a crosslinked or stabilized liposome or lipid vesicle having antigen encapsulated therein.

26. The method of claim 25 wherein the carrier is targeted using antibody or antibody fragment or ligand for molecules specific to or preferentially expressed on the surface of the antigen presenting or phagocytic cells.

27. The method of claim 22 wherein the carrier is formed of a synthetic polymeric carrier.

28. The method of claim 27 wherein the carrier is biodegradable by enzymes or hydrolysis.

29. The method of claim 22 wherein the antigen is an allergen which can crosslink IgE and induce anaphylaxis and the composition is administered to induce a greater immune response without causing anaphylaxis at a dosage greater than antigen can be administered in an unencapsulated form without increasing the risk of anaphylaxis.

30. The method of claim 29 wherein the antigen is selected from the group consisting of egg proteins, soybean proteins, peanut proteins, latex rubber proteins, milk proteins, wheat proteins, fish, crustaceans, tree nuts, and insect venom proteins.

31. The method of claim 22 wherein the composition induces tolerance in less time than through administration of unencapsulated antigens over a prolonged period of time.

32. The method of claim 22 wherein the composition is administered to an individual with an autoimmune disease.

33. The method of claim 22 wherein the carrier releases antigen in response to low pH after phagocytosis or endocytosis.

34. The method of claim 22 wherein the composition is administered by injection.

35. The method of claim 22 wherein the composition is administered by local or topical application to a mucosal surface.

36. The method of claim 22 wherein the composition is formulated to induce a T helper cell 1 response or suppress a T helper cell 2 response.

37. The method of claim 36 wherein the composition induces a T helper cell 1 response and comprises a cytokine selected from the group consisting of IL-2, IL-12, IL-18, IFN-gamma, and TNF.

38. The method of claim 36 wherein the composition suppresses a T helper 2 response and comprises an antagonist or inhibitor of a cytokine selected from the group consisting of IL-4, IL-5, IL-6, IL-10, and IL-13.

39. The method of claim 22 comprising an adjuvant or wherein the carrier is formed of materials acting as a adjuvant.
